(19) United States
(12) Patent Application Publication
(10) Pub. No.: US 2004/0028553 A1
(43) Pub. Date: Feb. 12, 2004

(54) Use of pulsed light to deactivate toxic and pathogenic bacteria

(75) Inventor: Louis R. Panico, Danvers, MA (US)

Correspondence Address: HALE AND DORR, LLP, 60 STATE STREET, BOSTON, MA, 02109

(73) Assignee: Xenon Corporation

(21) Appl. No.: 10/319,102

(22) Filed: Dec. 13, 2002

Related U.S. Application Data
(60) Provisional application No. 60/340,693, filed on Dec. 13, 2001.

(51) Int. Cl. A61L 2/10

(52) U.S. Cl. 422/24; 250/455.11

(57) ABSTRACT

A system and method are used to deactivate bacteria on articles such as pieces of mail or keyboards. With mail, the pulses are sufficient to destroy a substantial amount of the bacterial without also removing inks or other indicia from the mail.

### CROSS-REFERENCE TO RELATED APPLICATION

This application claims priority from Provisional Application Serial No. 60/340,693, filed Dec. 13, 2001, which is incorporated herein by reference.

### BACKGROUND

The prevention of contamination by bacteria, such as Bacillus anthracis, is an important issue with respect to objects, including common personal items such as mail and keyboards. Bacterial endospores, of the type produced by Bacillus and Clostridium species, are known to be highly resistant to various forms of radiation and other physical and chemical agents.

Pathogenic organisms manufactured for warfare or attack upon civilian populations can be artificial and differ significantly from naturally occurring pathogens. Artificial pathogens may be grown or manufactured in laboratories under conditions and in the presence of chemicals and/or nutrients that are different from those in which they reproduce and grow in their natural environment. Spores can be “weaponized” by adding chemicals that disperse the spores more readily and confer traits or properties that allow these organisms to survive during various methods of distribution in air, water or by solid objects. Manufacturing the biological warfare pathogens under these conditions can improve the stability of the pathogens to physical and chemical agents of decontamination. Because of these alterations, conventional methods of decontamination or inactivation of naturally occurring pathogens are not obvious choices and a guarantee of equal effectiveness.

There is a need for a simple, yet effective method of deactivating such bacteria that may be found on mail, keyboards, and other objects.

### SUMMARY OF THE INVENTION

The present invention includes a method and a pulsed-UV system that can successfully decontaminate pathogens used in biowarfare as demonstrated by inactivating a biological indicator artificially produced to be one of the most resistant organisms to conventional methods of decontamination and is thought to be similar to biowarfare spores.

### BRIEF DESCRIPTION OF DRAWINGS

FIGS. 1-6 are graphs of samples of bacterial decontamination under various conditions.

FIG. 7 is a block diagram of a system for deactivating bacteria.

### DETAILED DESCRIPTION

Bacteria can be deactivated through the use of high intensity pulsed ultraviolet (UV) light. The UV light generated by xenon lamps in a pulsed system mode rapidly and effectively renders pathogenic (disease causing) microorganisms incapable of reproducing. Two or three pulses within one second has been demonstrated to be sufficient to kill all or a very large percentage of bacterial spores.

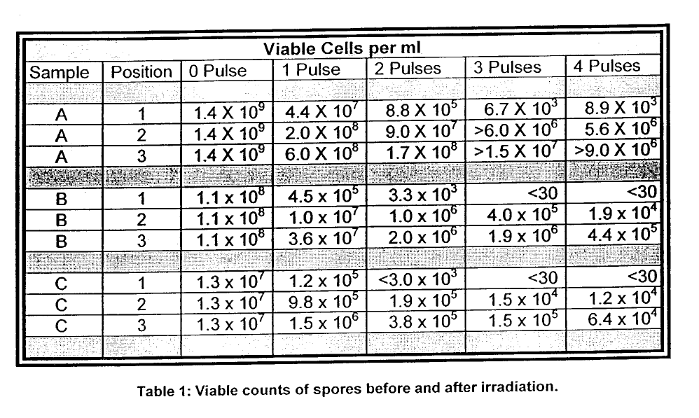

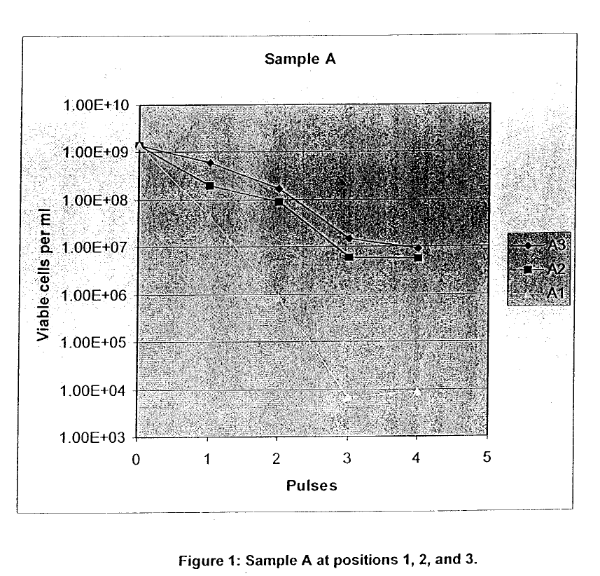

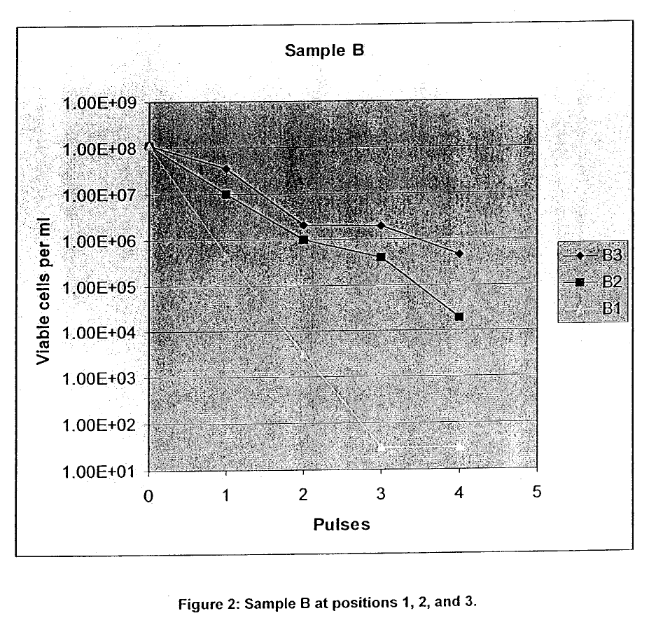

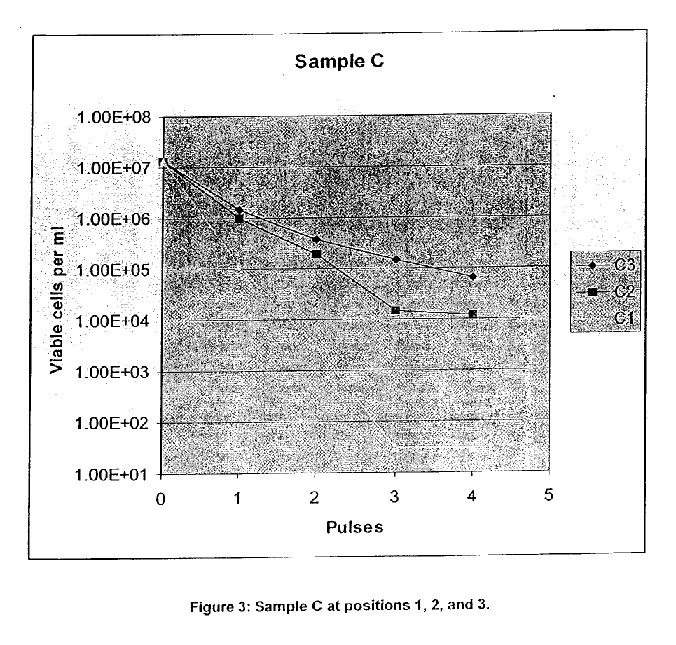

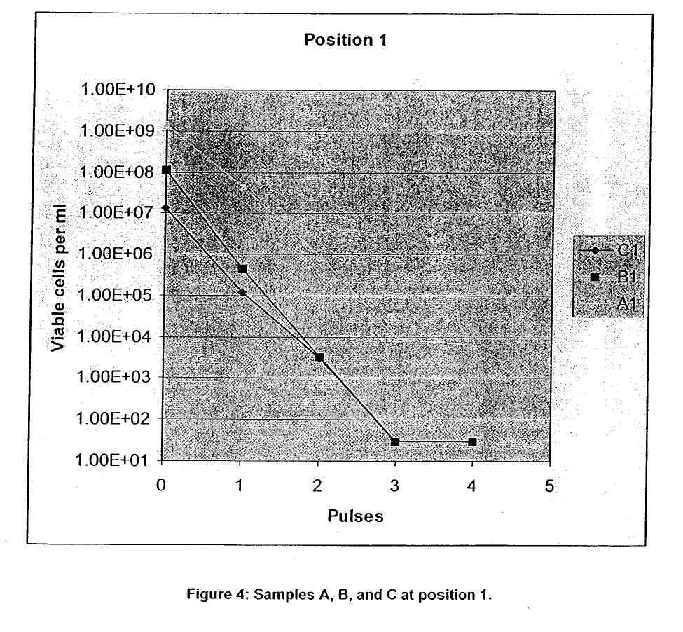

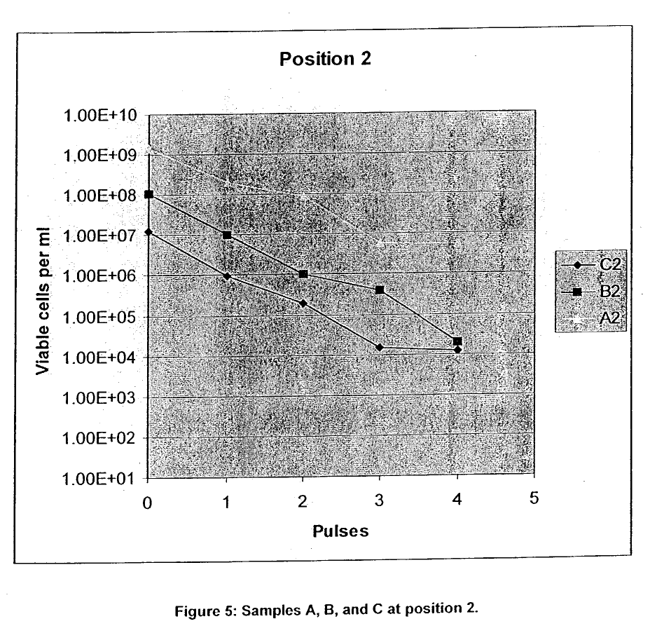

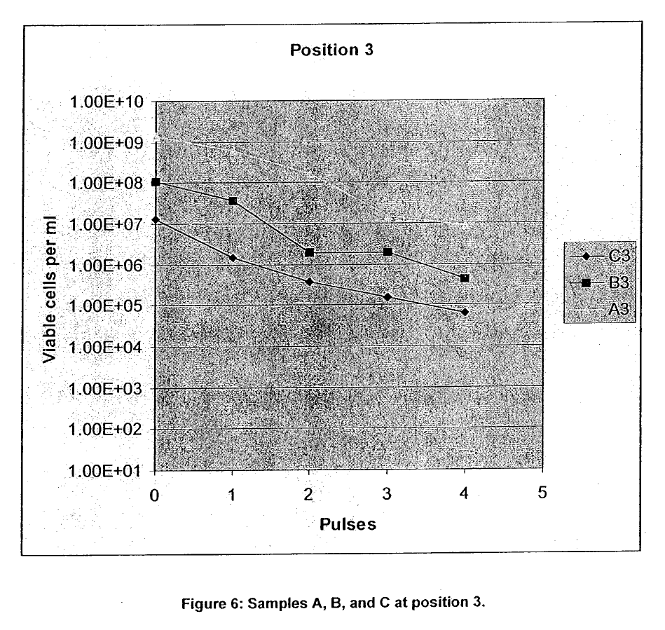

As indicated in the example below and in FIGS. 1-6 and Table 1, the pulsed UV system described herein was found to be highly effective for Bacillus subtilis, which is accepted as a substitute model for bacterial endospores of the type produced by Bacillus and Clostridium species.

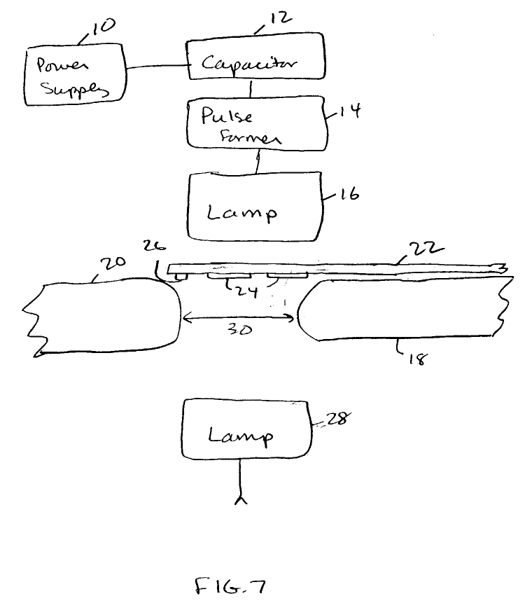

Referring to FIG. 7, an embodiment of a system according to the present invention includes a power supply 10, energy storage (capacitor) 12, pulse former 14, and lamp assembly 16 with related optics. These components are generally known, including in a SteriPulse XL-3000 system provided by Xenon Corporation. Lamp assembly 16 concentrates a high intensity, short duration (each as set out below) UV light pulse to a workpiece to be sterilized.

In addition, the SteriPulse XL-3000 can be integrated with conveyors 18, 20 and other handling devices to input items to and through the sterilization system, and for unloading. For example, a first conveyor 18 can transport the mail, such as piece 22 with writing 24 (or other object), to a second conveyor and past a sterilizing head where one or several xenon pulsed lamps are then activated.

An object, such as a piece of mail, can have put on it a material 26 that changes color in the presence of ultraviolet light to serve as an indicator that the mail has been treated. The material can be put on as a dot, a line, or other suitable indicia. Materials that change color in response to ultraviolet light are generally known and include spiroxazine compounds, spiropyran compounds, spiro-induline compounds, thiopyran compounds, benzopyran compounds, benzothioxanthone oxides, and others (see, e.g., U.S. Pat. No. 6,245,711, which is incorporated herein by reference).

A second lamp 28 can be located under conveyors 18, 20 at a gap 30 formed therebetween. The gap is sufficiently small relative to the lengthwise and widthwise directions of the mail to allow the mail to stay on the conveyors, while the light can access the piece of mail 22 (or keyboard, or other object) through the gap. This system also allows the second conveyor to remain substantially “cleaner” than the first conveyor.

Lamp 28 can be independently controlled with a separate power supply capacitor and pulse former, or can have some of these components shared, as described in WO 02/090114 which is incorporated herein by reference in its entirety. The pulses can be provided simultaneously or in an alternating manner, or in a variety of configurations as described in the incorporated patent publication.

The system can have an untreated bin of objects, such as mail, to first conveyor 28, and a second treated bin from second conveyor 30, all arranged in a compact manner.

Exemplary settings and positions are described in the example below, although the configuration of the device can be altered for this application. The example used a linear lamp, although other shapes (like spiral), numbers of lamps, and settings could be used:

Range of Operating Parameters:

Pulse Duration: 0.1-1,000 microseconds measured at ⅓ peak energy.

Energy per Pulse: 1-2,000 joules.

Pulse Recurrence Frequency: Single Pulse or one (1) to one thousand (1,000) pulses per second.

Exposure Interval: 0.1 to 1000 seconds, or single pulse, or continuous pulsing.

Lamp Configuration: (shape): linear, helical or spiral design. Spectral Output: 100-1,000 nanometers.

Lamp Cooling: ambient, forced air or water.

Wavelength Selection: (external to the lamp):Broadband or optical filter selective.

Lamp Housing Window: quartz, suprasil, or sapphire for spectral transmission.

Sequencing: Burst mode, synchronized burst mode, or continuous running.

### EXAMPLE 1

Research Test Procedure

Four 2-L flasks, each containing 500 ml of DS medium (a nutrient broth-based growth and sporulation medium for Bacillus subtilis), were inoculated with B. subtilis strain SMY (a standard wild-type strain) and incubated with vigorous shaking for 36 hours at 37° C. Spore formation was verified microscopically. Spores were harvested by centrifugation and washed twice with sterile, deionized water. The stock of spores was stored in water at 4° C.

The spore stock was diluted in sterile, deionized water to give concentrations of approximately 1×10⁹, 1×10⁸, and 1×10⁷ spores per ml, which were the concentrations of Samples A, B, and C, respectively. Fifty-microliter samples of each dilution were placed at three different locations with respect to the UV source and irradiated with 1 to 4 pulses of light. The samples were recovered, diluted as necessary with sterile water, and spread on agar plates containing a nutrient medium that supports growth of B. subtilis. After overnight incubation at 30° C., the colonies that arose were enumerated. Based on the number of colonies obtained at a given dilution of the irradiated spores, the surviving titer for each sample was calculated.

The UV source was a SteriPulse XL-3000 System provided by Xenon Corporation. The samples were placed as follows under an elongated lamp with a lamp axis along the elongated direction, and the midpoint referring to a central point along the length and width.

Position 1—at the lamp axis and at the midpoint of the lamp.

Position 2—1 cm off the lamp axis and at the midpoint of the lamp.

Position 3—1 cm off the lamp axis and 6.8 inches (172 mm) to the side of the midpoint of the lamp.

The energy per pulse was about 505 Joules, with a pulse duration of 320 microseconds.

As shown in the accompanying table and figures, the killing of spores was observed for all dilutions of the spore preparation at all positions with respect to the axis and midpoint of the lamp. Deactivation was most effective, however, when the sample was on the lamp axis and at the midpoint of the lamp. The kill rate was similar for all dilutions at a given position, although the most concentrated suspension may be killed slightly less effectively. Borne out by further experiments, such a result might imply that spores shield each other when they are above a certain concentration.

Microscopic analysis after irradiation (Sample A, 4 pulses) revealed that most of the spores had disintegrated.

Conclusions included the following:

1. The SteriPulse XL-3000 System is an effective device for reducing the viability of B. subtilis spores in suspension. Killing is rapid (1 second or less) and reduces viability by a significant factor. Starting with spore suspensions at 1×10⁸ (Sample B) or 1×10⁷ spores (Sample C) per ml, it was possible to eliminate viability with three pulses of UV light in 1 second.

2. The most concentrated sample, 1×10⁹ spores per ml (Sample A), was reduced in viability by 100,000-fold with three pulses.

3. Killing at Position 1 was much faster than at Positions 2 and 3. Thus, the most effective sanitization occurs on the lamp axis. Since there was only a small difference between the results obtained at Positions 2 and 3, it is likely that irradiation is equally effective across nearly the entire width of the lamp coverage.

4. Since other species of Bacillus and Clostridium are observed to exhibit similar responses to UV light, it is reasonable to infer that the methods described here would yield similar results with spores of other species, including Bacillus anthracis.

5. Results were obtained at the lower end of the energy range, and thus much more energy could be used.

### EXAMPLE 2

One problem with the use of pulsed light on mail is that the light can damage writing or bar codes. Writing can be hand-written ink or pencil, and other text can be printed in ink. A bar code would typically be printed with ink.

It has been shown here, however, that parameters can be selected to avoid deterioration in the ink, such that the writing remains clear and legible, and the bar code remains readable.

The following parameters for deactivation with the pulsed light treatment using Bacillus Subtilis (a surrogate of Bacillus Anthracis) were as follows:

A. The active treatment area (footprint) of the SteriPulse-XL 3000 was approximately 1″ (2.5 cm) wide by 14″ (35 cm) long—at 1″ (2.5 cm) from the treatment surface.

B. Pulse rate: 3 pps (pulses per second)

C. Electrical energy: 505.4 joules per pulse

D. Pulse duration: 320 microseconds

E. Effective spore reduction (static test) was at <1 second or 3 pps at the target area

F. The total optical energy delivered to the target was 1.27 j/cm2 per pulse

G. Therefore the transfer speed of the conveyor would be 1 in/sec (2.5 cm/sec)

There was no indication of damage to the envelope addresses or barcodes during tests using the parameters above. Thus it was determined that sufficient energy could be employed to substantially deactivate the bacteria by at least a factor of 1000, 10,000, 100,000, or more.

It is believed that these parameters could be varied by ±50% in combinations to have sufficient energy. Energy levels over 1000 J per pulse, however, might not work.

Having described embodiments of the present invention, it should be apparent that modifications can be made without departing from the scope of the invention as defined by the appended claims.

1. A method comprising providing to a surface of an object with ink indicia thereon a series of ultraviolet light pulses with sufficient energy to deactivate bacteria thereon by a factor of 1000 or more, while maintaining the readability and/or machine detectability of the indicia.

2. The method of claim 1, wherein providing the pulses is performed to one or pieces of mail that include handwriting.

3. The method of claim 1, wherein providing the pulses is performed to one or pieces of mail that include a barcode.

4. The method of claim 1, further comprising transporting a plurality of objects with ink indicia thereon along a first conveyor to a second conveyor, the first and second conveyors defining a gap therebetween that is smaller than a lengthwise or widthwise direction of the objects, wherein a lamp for providing the pulses is located for providing a pulse of light through the gap.

5. The method of claim 4, wherein the objects are pieces of mail.

6. The method of claim 4, further comprising providing a lamp on another side of the conveyors, so that at least two lamps are used to provide pulses on opposite sides of the

7. The method of claim 1, wherein by the bacteria is one of the Bacillus and Clostridium species.

8. A system comprising:
a first conveyor for transporting articles;
a second conveyor for transporting articles received from the first conveyor, and spaced from the first conveyor by a gap;
a lamp for providing light energy to deactivate bacterial spores on articles transported on the conveyor, the lamp being located to provide light between the conveyors, and through the gap to the articles.

9. The system of claim 8, wherein the lamp is an ultraviolet light pulse lamp which provides a series of high-energy, short-duration pulses to the articles.

10. The system of claim 8, wherein bacterial spores on the articles are reduced by a factor of 10³.

11. The system of claim 8, wherein bacterial spores on the articles are reduced by a factor of 10⁴.

12. The system of claim 8, wherein bacterial spores on the articles are reduced by a factor of 10⁵.

13. The system of claim 8, wherein bacterial spores on the articles are reduced by a factor of 10⁶.

14. The system of claim 8, wherein the bacteria is one of the Bacillus and Clostridium species.

15. The system of claim 8, further comprising a second lamp, such that the lamp for providing light between the conveyors and the second lamp are on opposite sides of the articles.

16. The system of claim 15, wherein the articles include paper.

17. The system of claim 16, wherein the articles include pieces of mail.

18. The system of claim 15, wherein the articles include pieces of mail.
